(19) United States
(12) Patent Application Publication
(10) Pub. No.: US 2003/0055381 A1
(43) Pub. Date: Mar. 20, 2003

(54) Medical devices

(76) Inventor: Bradley M. Wilkinson, North Haledon, NJ (US)

Correspondence Address: BECTON, DICKINSON AND COMPANY, 1 BECTON DRIVE, FRANKLIN LAKES, NJ, 07417-1880, US

(21) Appl. No.: 10/228,798

(22) Filed: Aug. 27, 2002

Related U.S. Application Data
(60) Provisional application No. 60/323,186, filed on Sep. 17, 2001.

(51) Int. Cl. A61M 5/32

(52) U.S. Cl. 604/177; 128/919

(57) ABSTRACT

The present invention is directed to a shieldable blood collection set. The shieldable blood collection set includes a fixture for connecting the blood collection set to a receptacle, an intravenous needle assembly including a needle cannula having a puncture tip, and a flexible tube extending between the fixture and the intravenous needle assembly. A shield assembly is in engagement with the needle assembly. The shield assembly is adapted for movement from a first position to a second position for protectively shielding the puncture tip of the needle cannula. The flexible tube includes a plurality of helical turns establishing a coiled structure. Such a coiled structure provides the blood collection set with an enlarged surface area for grasping by the palm of a user, thereby facilitating one-handed activation of the safety shield assembly.

This application claims priority of U.S. Provisional Patent Application No. 60/323,186 filed Sep. 17, 2001

### BACKGROUND OF THE INVENTION

1. Field of the Invention

The present invention relates to blood collection sets for safe and convenient handling of needles. More particularly, the present invention relates to a blood collection set including a needle assembly having a safety shield which is easily manipulated by a user.

2. Description of Related Art

Disposable medical devices having piercing elements are typically used for administering a medication or withdrawing a fluid, such as blood collecting needles, fluid handling needles and assemblies thereof. Current medical practice requires that the fluid containers and needle assemblies used in such systems be inexpensive and readily disposable. Consequently, existing blood collection systems, for example, typically employ some form of durable, reusable holder on which detachable and disposable needles and fluid collection tubes may be mounted. A blood collection system of this nature can be assembled prior to use and then disassembled after usage. Thus, these blood collection systems allow repeated use of the relatively expensive holder upon replacement of the relatively inexpensive needle and/or fluid collection tube. In addition to reducing the cost of collecting blood specimens, these blood collection systems also help minimize the production of hazardous medical waste.

A blood collection set or intravenous (IV) infusion set typically includes a needle cannula having a proximal end, a pointed distal end and a lumen extending therebetween. The proximal end of the needle cannula is securely mounted in a plastic hub with a central passage that communicates with the lumen through the needle cannula. A thin flexible thermoplastic tube is connected to the hub and communicates with the lumen of the needle cannula. The end of the plastic tube remote from the needle cannula may include a fixture for connecting the needle cannula to a blood collection tube or some other receptacle. The specific construction of the fixture will depend upon the characteristics of the receptacle to which the fixture will be connected.

In order to reduce the risk of incurring an accidental needle-stick wound, protection of used needle tips becomes important. With concern about infection and transmission of diseases, methods and devices to enclose the used disposable needle have become very important and in great demand. For example, needle assemblies commonly employ a safety shield that can be moved in a forward position into shielding engagement with a used needle cannula without risking an accidental needle stick.

Some needle shields are referred to as tip guards, and include a small rigid guard that can be forwardly telescoped along the length of a needle cannula and extended over the puncture tip of the needle for protection. Such conventional tip guard may include some form of tether for limiting the travel of the tip guard to the length of the needle cannula. Additionally, such conventional tip guard typically includes structure that lockingly engages over the tip of the used needle cannula to prevent a re-exposure of the needle. The structure for preventing re-exposure may include a metallic spring clip or a transverse wall integrally formed with one end of the tip guard.

Shielding of the needle in such assemblies typically involves grasping the needle hub with the flexible tubing in one hand and manipulating the needle shield into a forward blunting position with the other hand. Accordingly, activation of needle shields, and in particular forwardly blunting needle shields, typically requires two hands in order to properly shield the needle. Moreover, the small diameter of the flexible tubing attached to the needle assembly does not provide an adequate surface for handling.

Accordingly, a need exists for a needle assembly for use with a blood collection set which achieves secure and effective shielding of a used needle tip and which is easy to operate with a single hand.

### SUMMARY OF THE INVENTION

The present invention is directed to a blood collection set including a shieldable needle device. The shieldable blood collection set includes a fixture for connecting the blood collection set to a receptacle, an intravenous needle assembly including a needle cannula having a puncture tip, and a flexible tube extending between the fixture and the needle assembly. A shield assembly is in engagement with the needle assembly. The shield assembly is adapted for movement from a first position to a second position for protectively shielding the puncture tip of the needle cannula. The flexible tube includes a first end connected to the fixture and an opposed second end connected to the needle assembly, with a plurality of helical turns establishing a coiled structure between the fixture and the needle assembly. The coiled structure may include a circular cross-section, or may include an elliptical cross-section.

Desirably, the needle assembly includes a shield assembly adapted for movement from a first position to a second position for protectively shielding the puncture tip of the needle cannula. The shield assembly may include a housing extending about the needle cannula, and adapted for movement from the first position to the second position for protectively shielding the puncture tip of the needle cannula. The housing may include a lip portion adapted for engagement with a user's thumb for effecting movement from the first position to the second position.

Also, the needle assembly may include a hub, with the needle cannula extending from one end of the hub and the flexible tube attached to an opposing end of the hub. Such a hub desirably includes a pair of wings extending laterally from opposing sides thereof. The housing of the shield assembly may further include grooves extending laterally through the housing for accommodating the wings of the hub therethrough.

The coiled structure is desirably adjacent the shield assembly such that the shield assembly can be moved to the second position by a user's hand while maintaining the coiled structure in the palm of the user's hand. In this manner, one-handed activation of the safety shield assembly can be accomplished through the enlarged surface area established through the coiled structure for grasping by the palm of a user.

### DESCRIPTION OF THE DRAWINGS

FIG. 1 is a perspective view of a fully assembled blood collection set in accordance with the present invention with a packaging cover thereon;

FIG. 2 is a top plan view of the shieldable needle assembly of the blood collection set of FIG. 1 in a retracted position;

FIG. 3 is a cross-sectional view taken along line 3-3 of FIG. 2;

FIG. 4 is a cross-sectional view in an alternate embodiment;

FIG. 5 is a perspective view of the blood collection set shown in use; and

FIG. 6 is a perspective view of the shieldable needle assembly in an extended shielded position.

### DETAILED DESCRIPTION

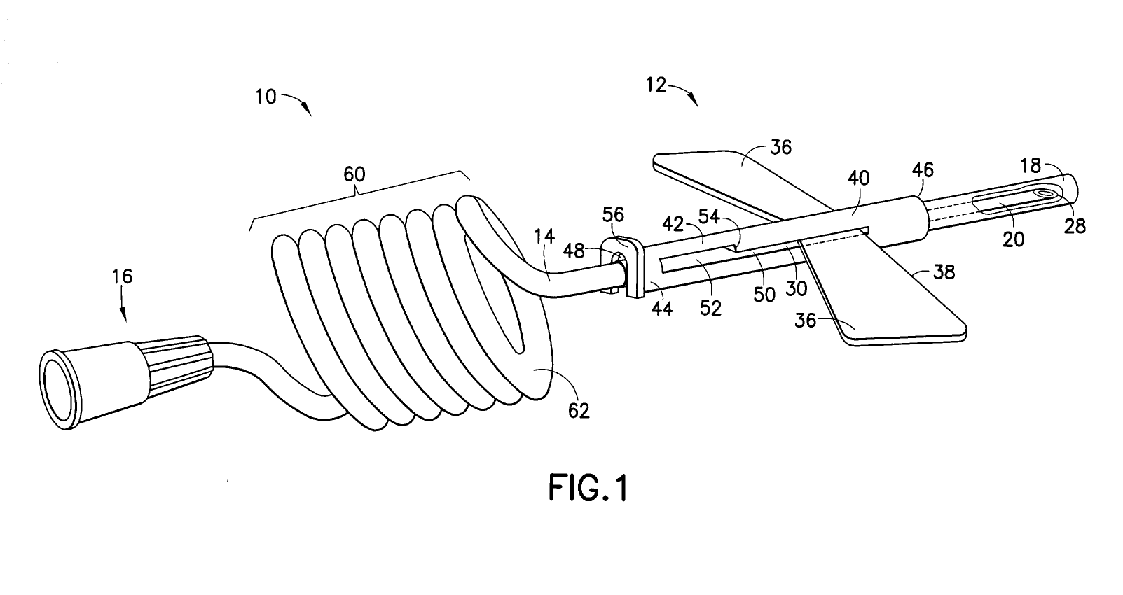

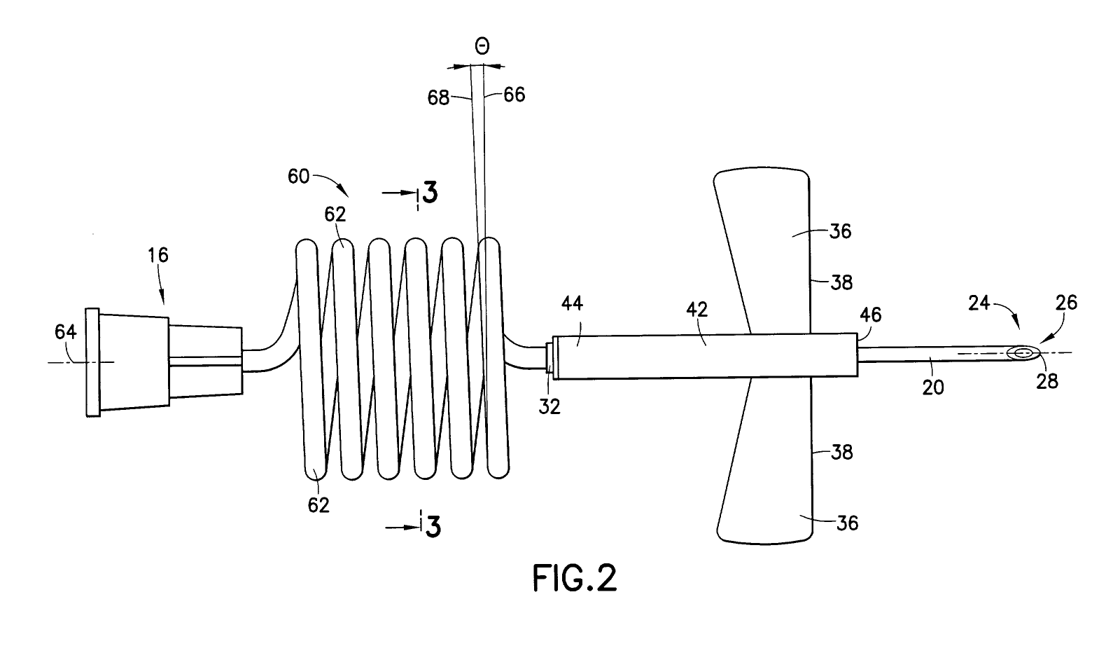

Referring to the drawings in which like reference characters refer to like parts throughout the several views thereof, FIGS. 1-6 illustrate a blood collection set in accordance with the present invention and the related features. As shown in FIGS. 1 and 2, blood collection set 10 includes a shieldable needle device 12, a flexible tube 14 extending from needle device 12, a fixture 16 mounted to tube 14 and a packaging cover 18 removably mounted to portions of needle device 12 opposite tube 14, such as through a frictional engagement.

Shieldable needle device 12 may be any needle device including means for shielding an exposed needle tip, as is known in the art. Desirably, needle device 12 is a forward shielding needle device, in which a shielding member is moved from a first position in which the tip of the needle is exposed, to a second position encompassing the tip of the needle to shield the needle tip. For example, as shown in FIGS. 1 and 2, shieldable needle device 12 of blood collection set 10 includes a needle cannula 20, a hub 30, and a shield assembly 40. While the present invention is described in terms of a blood collection set incorporating needle device 12, it is understood that various other needle devices may be apparent to those skilled in the art for use in connection with the blood collection set of the present invention.

Needle cannula 20 includes a proximal end and an opposing distal end 24, with lumen 26 extending through needle cannula 20. Distal end 24 of needle cannula 20 is beveled to define a sharp puncture tip 28, such as an intravenous puncture tip. Puncture tip 28 is provided for insertion into a patient's blood vessel, such as a vein, and is therefore designed to provide ease of insertion and minimal discomfort during venipuncture.

Needle assembly 12 further includes hub 30. Hub 30 is a unitary structure, desirably molded from a thermoplastic material. Hub 30 is defined by a rigid tubular wall 32 extending from a proximal end to distal end thereof. Tubular wall 32 is characterized by an internal passage extending therethrough from the proximal end to the distal end of hub 30.

Hub 30 further includes a pair of stabilizers in the form of wings 36 extending laterally from tubular wall 32 at opposing sides thereof. Wings 36 provide hub 30, and needle assembly 12, as a butterfly-type wing assembly, assisting in positioning and placement of needle assembly 12 and blood collection set 10 during a blood collection procedure.

Needle cannula 20 is positioned within the internal passage of hub 30, and extends from the distal end of hub 30. Desirably, needle cannula 20 and hub 30 are separate parts which are fixedly attached and secured through an appropriate medical grade adhesive or the like.

Needle assembly 12 further includes shield assembly 40. Shield assembly 40 is slidably telescoped for axial movement along needle assembly 12 between a first proximal position, in which needle cannula 20 is at least partially exposed, and a second distal position adjacent puncture tip 28, in which needle cannula 20 and puncture tip 28 are encompassed by shield assembly 40.

Shield assembly 40 includes a housing 42. Housing 42 is a unitary structure, desirably molded from a thermoplastic material, including a proximal end 44, a distal end 46, and an internal passage 48 extending between the ends. Shield assembly 40 further includes a pair of grooves 50 extending through housing 42 along opposing sides thereof. Grooves 50 are adapted to accommodate wings 36 of hub 30 extending through housing 42, during movement of shield assembly 40 from the first position to the second position. Grooves 50 of shield assembly 40 further include an enlarged portion, such as slots 52, adjacent proximal end 44 of housing 42 and extending through housing 42. Shield assembly 40 further includes a lip 56 at a proximal end thereof. Lip 56 provides an extending surface for engagement with a user's thumb in order to activate shield assembly 40 for forward movement between the first position and the second position, as will be discussed.

It is noted that shield assembly 40 may be any type of shield assembly which is capable of forward movement to a shielding position to encompass needle cannula 20. In particular, shield assembly 40 may include any blunting or shielding mechanism, which is adapted for movement along needle cannula 20 when shield assembly 40 is moved from the proximal position to the distal position, and which extends over puncture tip 28 of needle cannula 20 when shield assembly 20 is moved to the fully shielded distal position.

For example, housing 42 of shield assembly 40 may include an end cap having a blocking surface that is slidably telescoped on needle cannula 20 for axial movement from the proximal position to the distal position, where the end cap protectively surrounds distal end 24 of needle cannula 20. Additionally, the end cap may be configured to prevent proximal movement after the blocking surface of the end cap has advanced sufficiently in a distal direction to protectively enclose puncture tip 28 of needle cannula 20. The end cap may be in the form of a tip guard, which may include a tip guard housing formed from a plastic material, and a protective clip, such as a metallic spring clip mounted to the housing. In such an arrangement, the clip can be biased against the needle cannula when the tip guard is in the proximal position and can be resiliently moved over the distal end and distal tip of the needle cannula when the tip guard is in the distal position, thereby preventing piercing by the tip of the needle. Other shield assemblies will be apparent to those skilled in the art.

As noted, blood collection set 10 further includes flexible tube 14 extending between needle device 12 and fixture 16. Flexible tube 14 includes a first end connected to needle device 12, and a second end connected to fixture 16. Fixture 16 is provided for connecting blood collection set 10 to a receptacle, for example a non-patient needle assembly and blood collection tube, as is known in the art.

Flexible tube 14 further includes a coiled structure in the form of coil 60. In particular, flexible tube 14 includes a plurality of turns 62 extending helically from one end of flexible tube 14 to the other end. Such plurality of helical turns 62 provides flexible tube 14 with an enlarged surface area, thereby defining the shape of coil 60. Such an enlarged surface area provides for better tactile engagement with the user's hand during activation of shield assembly 40, as will be discussed in further detail herein.

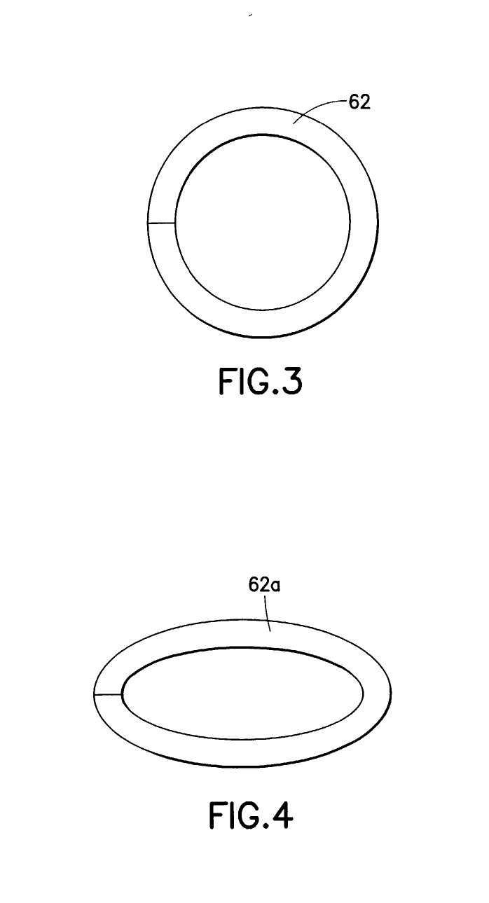

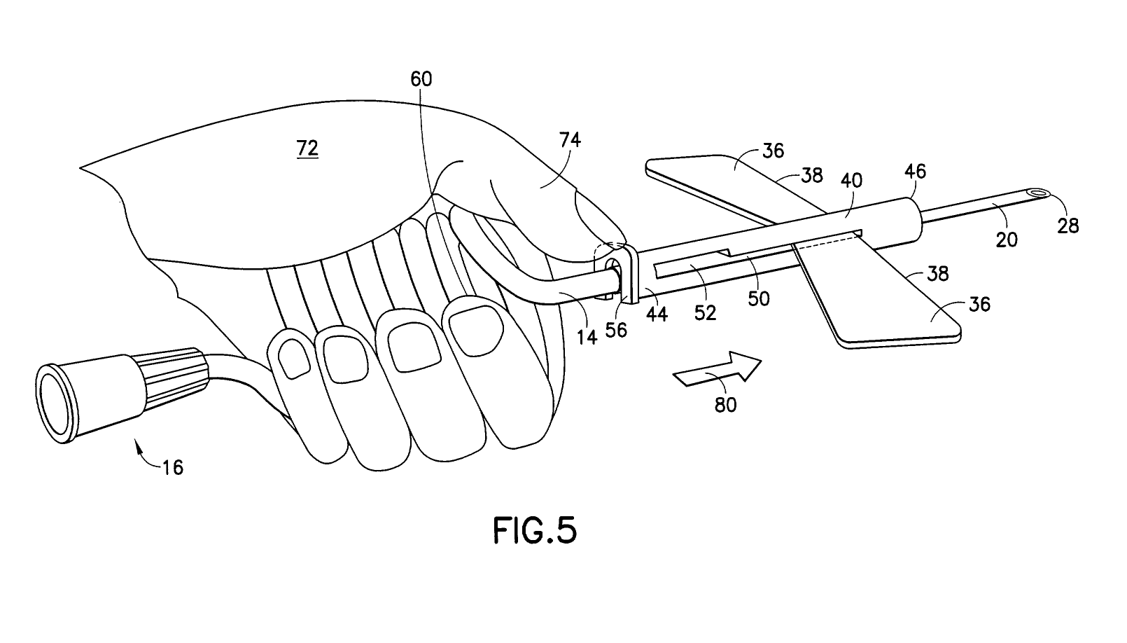

FIG. 3 depicts a cross-section of one turn 62 of coil 60. As can be seen, coil 60 includes a substantially circular cross-section, providing coil 60 with an enlarged surface area as compared with the diameter of a linear portion of flexible tube 14 in an uncoiled state. Moreover, FIG. 2 demonstrates the helical nature of turns 62 of coil 60. In particular, flexible tube 14 coils about axis 64 of blood collection set 10, defining coil 60 extending generally axially along axis 64 of blood collection set 10. Each of turns 62 extends circumferentially about axis 64, in a generally helical direction in a series of planes which are substantially parallel to a cross-section of axis 64. The helical nature of coil 60 is defined by angle θ, which defines the slope of the helical turns with respect to line 66, which represents a perpendicular cross-section of axis 64. When angle θ is a small angle approaching 0° and helical turns 62 are tightly wound, coil 60 includes a generally circular cross-section, as depicted in FIG. 3. Moreover, when angle θ is a larger angle increasing away from 0°, for example, toward 45°, a circular cross-sectional profile can be maintained, if helical turns 62 are spaced apart from each other and not maintained in a tightly wound manner in contact with each other. On the other hand, when angle θ is a larger angle increasing away from 0°, for example, toward 45°, and helical turns 62 remain tightly wound or in contact with each other, the slope of coil 60 increases, with coil 60 including a generally elliptical cross-section, as depicted in the alternate embodiment of FIG. 4. Such an elliptical cross-section provides coil 60 with a generally tubular elliptical profile for grasping by a user's hand. Such an elliptical profile can alternatively be provided by providing the coil in a generally oval helix, as opposed to a generally circular helix.

Activation of shield assembly 40 is accomplished by exerting a force on housing 42 in the direction of arrow 80 while maintaining hub 30 (and needle cannula 20 attached thereto) in place, thereby causing housing 42 to move along needle cannula 20 in the direction of arrow 80 toward distal end 24 and toward puncture tip 28. Wings 36 of hub 30 extend through housing 42 of shield assembly 40. During such movement of housing 42 along needle cannula 20, wings 36 slide within grooves 50 of housing 42. As such, housing 42 moves from the proximal position to the distal position. When in the distal position, wings 36 travel within grooves 50 to slots 52, which represent an enlarged section of grooves 50. Slots 52 include a shoulder 54 on a distal edge thereof. Once wings 36 are positioned within slots 52, the forward edge 38 of wings 36 abuts shoulder 54 of slots 52 in an interference engagement. As such, shield assembly 40 is prevented from moving from the distal position to the proximal position, thus providing a locking mechanism for maintaining needle device 12 in the shielded position.

Shield assembly 40 may require active exertion of force thereon in order to effect movement of housing 42 from the proximal position to the distal position in the direction of arrow 80. For example, a force may be exerted by the user on lip 56 of housing 42 in the direction of arrow 80. Such force causes housing 42 to move with respect to hub 30 in a direction toward distal end 24 of needle cannula 20, i.e., between a proximal position and a distal position. In particularly desirable embodiments, needle device 12 is passively shieldable, in that housing 42 automatically moves in a direction of arrow 80 to achieve secure shielding of puncture tip 28 upon release of housing 42 from the proximal position. To achieve such passive shielding, shield assembly 40 may include means for storing energy for movement of shield assembly 40 in a direction toward distal end 24 of needle cannula 20 upon release of housing 42 from the proximal position. For example, shield assembly 40 may include a torsion spring capable of exerting a biasing force to propel housing 42 from the proximal position to the distal position. Housing 42 may be releasably maintained in the proximal position through a mechanism such as a latch, which can be released to permit movement of housing 42 from the proximal position to the distal position. Desirably, the means for storing energy for movement of shield assembly 40 automatically engages to move shield assembly 40 once a force is exerted upon housing 42 in a direction of arrow 80, such as through a user's thumb.

Blood collection set 10 can be packaged substantially in the condition shown in FIG. 1. Coil 60 provides blood collection set 10 with a profile which reduces packaging as compared with a conventional blood collection set, thereby reducing costs associated with packaging and handling of blood collection sets.

Prior to use, blood collection set 10 is removed from its package. Blood collection set 10 is provided with needle device 12 assembled and including flexible tube 14 extending from needle device 12 and connected to fixture 16, with coil 60 in a tightly wound profile. After removing blood collection set 10 from its package, it can be assembled with other appropriate medical equipment for use. For example, fixture 16 may be connected to an appropriate receptacle for providing fluid communication with lumen 26 through needle cannula 20, such as a non-patient needle assembly and a needle holder for use with blood collection tubes, as are known in the art.

To prepare for use of blood collection set 10, the user grasps blood collection set 10, with coil 60 held in the user's palm, and with needle device 12 maintained between the user's thumb and forefinger. Packaging cover 18 is then grasped and urged distally to disengage from needle cannula 20, thereby exposing puncture tip 28 of needle cannula 20.

The medical practitioner can then urge puncture tip 28 at distal end 24 of needle cannula 20 into a targeted blood vessel of a patient, while needle device 12 is maintained between thumb and forefinger to assist in controlled entry by the medical practitioner. During such positioning, wings 36 may be bent inwardly toward each other between the user's thumb and fingers.

After the targeted blood vessel has been accessed, the medical practitioner can release the grip. Wings 36 may then be secured to the user's skin, such as through tape, to prevent movement of needle device 12 during the procedure. The coiled nature of coil 60 of flexible tube 14 assists in preventing needle cannula 20 from accidentally pulling out of the patient's vessel. For example, in conventional blood collection systems, there is little slack in the tube extending from the needle device. As such, any movement of the patient, or extension of the tube by the user, may result in the needle cannula accidentally pulling out of the patient. Coil 60 provides flexibility to tube 14, thereby providing more freedom of movement between the patient and the user. In addition, coil 60 maintains fixture 16 in a position adjacent the puncture site. As such, the receptacle attached to fixture 16 can be maintained adjacent the puncture site, providing for better operation by the user.

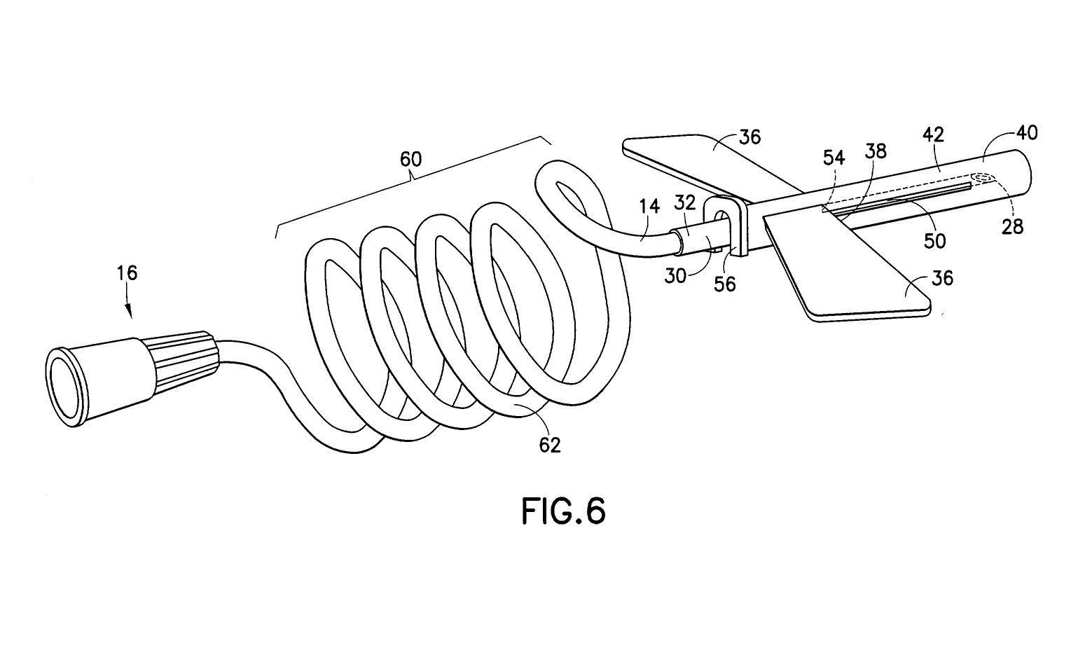

Upon completion of the procedure, such as when all desired samples have been drawn, needle cannula 20 is withdrawn from the patient. The user grasps blood collection set 10 about coil 60 as shown in FIG. 5, maintaining coil 60 within the user's hand 72, with the user's thumb 74 pressed against lip 56 of housing 42. After removal of needle cannula 20 from the patient, the user's thumb 74 is moved in a direction of arrow 80, thereby forcing housing 42 in a direction of arrow 80. The additional surface area provided to tube 14 through coil 60 provides blood collection set 10 with a profile which is particularly adaptable for fitting within the palm of a user's hand, thereby providing for simple and effective one-handed activation of the shield assembly.

During movement of housing 42 along needle cannula 20 toward the distal end 24, wings 36 slide through grooves 50, and into slots 52. Interference engagement between forward edge 38 of wings 36 and shoulder 54 of slots 52 prevents housing 42 from moving in the opposing direction. Shield assembly 40 has an overall dimension that will prevent movement of housing 42 distally beyond needle cannula 20. Hence, puncture tip 28 of needle cannula 20 is safely shielded. Blood collection set 10 may then be appropriately discarded. Coil 60 of flexible tube 14 provides blood collection assembly set 10 with an overall dimension that provides for ease of disposal as compared with conventional blood collection sets including extended flexible tubes.

While the needle assembly of the present invention has been described in terms of one embodiment for use in connection with a blood collection system, it is further contemplated that the needle assembly could be used with other medical procedures, such as in conjunction with a conventional intravenous infusion set, which are well-known in the art for use with needle assemblies.

While the present invention is satisfied by embodiments in many different forms, there is shown in the drawings and described herein in detail, the preferred embodiments of the invention, with the understanding that the present disclosure is to be considered as exemplary of the principles of the invention and is not intended to limit the invention to the embodiments illustrated. Various other embodiments will be apparent to and readily made by those skilled in the art without departing from the scope and spirit of the invention. The scope of the invention will be measured by the appended claims and their equivalents.

What is claimed:

1. A blood collection set comprising:
a fixture for connecting said blood collection set to a receptacle;
an intravenous needle assembly including a needle cannula having a puncture tip; and
a flexible tube extending between said fixture and said needle assembly, said flexible tube having a first end connected to said fixture and an opposed second end connected to said intravenous needle assembly, said flexible tube including a plurality of helical turns establishing a coiled structure.

2. A blood collection set according to claim 1, wherein said coiled structure includes a circular cross-section.

3. A blood collection set according to claim 1, wherein said coiled structure includes an elliptical cross-section.

4. A blood collection set according to claim 1, wherein said needle assembly includes a shield assembly adapted for movement from a first position to a second position for protectively shielding said puncture tip of said needle cannula.

5. A blood collection set according to claim 4, wherein said shield assembly comprises a housing extending about said needle cannula, said housing adapted for movement from said first position to said second position for protectively shielding said puncture tip of said needle cannula.

6. A blood collection set according to claim 5, wherein said housing includes a lip portion adapted for engagement with a user's thumb for effecting movement from said first position to said second position.

7. A blood collection set according to claim 1, wherein said needle assembly includes a hub, with said needle cannula extending from one end of said hub and said flexible tube attached to an opposing end of said hub.

8. A blood collection set according to claim 7, wherein said hub includes a pair of wings extending laterally from opposing sides of said hub.

9. A blood collection set according to claim 1, further comprising a packaging cover frictionally engaged on said needle cannula and securely surrounding said needle cannula.

10. A shieldable blood collection set comprising:
a fixture for connecting said blood collection set to a receptacle;
an intravenous needle assembly including a needle cannula having a puncture tip;
a shield assembly in engagement with said needle assembly, said shield assembly adapted for movement from a first position to a second position for protectively shielding said puncture tip of said needle cannula; and
a flexible tube extending between said fixture and said needle assembly, said flexible tube having a first end connected to said fixture and an opposed second end connected to said needle assembly, said flexible tube including a plurality of helical turns establishing a coiled structure.

11. A shieldable blood collection set according to claim 10, wherein said coiled structure is adjacent said shield assembly such that said shield assembly can be moved to said second position by a user's hand while maintaining said coiled structure in the user's hand.

12. A shieldable blood collection set according to claim 10, wherein said coiled structure includes a circular cross-section.

13. A shieldable blood collection set according to claim 10, wherein said coiled structure includes an elliptical cross-section.

14. A shieldable blood collection set according to claim 10, wherein said needle assembly includes a hub, with said needle cannula extending from one end of said hub and said flexible tube attached to an opposing end of said hub.

15. A shieldable blood collection set according to claim 14, wherein said hub includes a pair of wings extending laterally from opposing sides of said hub.

16. A shieldable blood collection set according to claim 15, wherein said shield assembly includes a housing, said housing having grooves extending laterally through said housing for accommodating said wings.

17. A shieldable blood collection set according to claim 10, further comprising a packaging cover frictionally engaged on said needle cannula and securely surrounding said needle cannula.
